(19) United States
(12) Patent Application Publication
(10) Pub. No.: US 2005/0131288 A1
(43) Pub. Date: Jun. 16, 2005

(54) Flexible, patient-worn, integrated, self-contained sensor systems for the acquisition and monitoring of physiologic data

(76) Inventors: Christopher T. Turner, Belmont, MA (US); Martin D. Wells, Needham, MA (US)

Correspondence Address: Mark J. Pandiscio;Pandiscio & Pandiscio, P.C., 470 Totten Pond Road, Waltham, MA, 02451-1914, US

(21) Appl. No.: 10/917,666

(22) Filed: Aug. 13, 2004

Related U.S. Application Data
(60) Provisional application No. 60/495,433, filed on Aug. 15, 2003.

(51) Int. Cl. A61B 5/04; A61B 5/00

(52) U.S. Cl. 600/391; 128/903; 600/300

(57) ABSTRACT

An apparatus and method for the transduction, acquisition, and processing of physiologic variables are described. The apparatus is a self-contained, patient-worn device consisting of a flexible substrate, a transduction means, a power generation means, and a signal acquisition and processing means, and a data transmission means.

### REFERENCE TO PENDING PRIOR PATENT APPLICATION

This patent application claims benefit to pending prior U.S. Provisional Patent Application Ser. No. 60/495,433, filed Aug. 15, 2003 by Christopher T. Turner et al. for INTEGRATED SENSOR FOR SLEEP DISORDER ASSESSMENT (Attorney's Docket No. GRAN-9 PROV), which patent application is hereby incorporated herein by reference.

### AREA OF THE INVENTION

The present invention relates to flexible, patient-worn, integrated, self-contained sensor systems for the acquisition and monitoring of physiologic data.

### BACKGROUND OF THE INVENTION

A large number of physiologic measurements are made using transducers that need to be in contact with the patient. Types of transducers utilized include electrical, mechanical, and chemical. Electrical physiologic transducers, electrodes, are used in the measurement of electromyograms (EMG), electroneurograms (ENG), electrocardiograms (ECG), electroencephalograms (EEG) to name a few. Mechanical transducers are used to record respiratory signals and patient activity. Chemical transducers are applied to determine the oxygen saturation of blood as an example.

These measurement modalities typically require each patient-worn transducer to be connected by at least one wire to a data acquisition instrument located near the patient. In applications such as polysomnography or high-density EEG, where many such transducers are used simultaneously, the number of individual wires connecting a patient to bedside monitoring equipment can be well over 100.

These wires are:
Difficult to apply.
Difficult to maintain throughout a measurement session.
Cumbersome to the patient and to the caregiver.
A source of measurement error and instability.
A deterrent to patient ambulation.

Furthermore, wired connections to physiologic transducers often need to be re-attached whenever the patient needs to leave the immediate vicinity of the external monitoring equipment or change their clothes.

### SUMMARY OF THE INVENTION

This patent application describes a platform medical technology that will provide a new means of assessing and treating a wide range of underlying conditions. The technology is based on the integration of all signal conditioning, data acquisition, digitization, signal processing, data analysis and external communication means with one or more transducers of physiologic information.

The basic technology is comprised of the following:
Flexible, adhesive backed substrate
An onboard power source, such as a thin-film, flexible battery or a more traditional small, rigid coin cell battery
A sensing mechanism (transducer)
Signal processing and/or control circuitry
   Either
A means of storing the sensed data, OR
A means of wirelessly communicating the sensed data

The device is primarily characterized by the fact that all power and electronics are integrated onto the flexible substrate and worn on the patient. This eliminates the need for a bulky instrument and for a tethered connection. The device is intended to be thin and flexible and to be patient worn in an unobtrusive manner. Several embodiments of the technology will be described that demonstrate the advantage of this technology over the state of the art.

### PREFERRED EMBODIMENT

Polysomonographic Sensor

Sleep apnea, the cessation of breath while sleeping, is a common and serious health problem. Four percent of middle-aged men and two percent of middle-aged women fit the criteria for sleep apnea. This corresponds with an NIH estimate of as many as 15 million Americans that are afflicted with the disorder, with a significant number of them undiagnosed. Untreated, sleep apnea is significant risk factor for serious health conditions such as hypertension and related cardiac disorders. In addition, the short term consequence of sleep apnea, a poor night's sleep, often leads to daytime sleepiness, causing lost productivity and increased automobile accidents. When diagnosed, apnea is usually effectively treated by a number of methods; the most common of which is continuous positive airway pressure (CPAP) treatment. Thus, there is a significant motivation to remedy the underdiagnosis of apnea and ensure that those with the condition receive treatment.

The gold standard for diagnosis of apnea is polysomnography (PSG), the monitoring and scoring of multiple physiologic parameters. Polysomnography is conducted while a patient sleeps overnight in a sleep lab under the supervision of polysomnographic technicians and sleep medicine physicians. Polysomnography typically involves the measurement of the following parameters: EEG, EOG, EMG, respiration, respiratory effort, EKG, pulse oximetry, body position and limb motion. A patient is connected to many individual sensors, electrodes, and leads to accomplish this testing. A polysomnographic amplifier is connected to the various sensors/leads and provides amplification and recording of the signals. Signals are review and scored according to standard criteria by a polysomnographic technician and sleep medicine physician. This scoring may result in a diagnosis of sleep apnea or related sleep disorders.

Although widely used and effective as a diagnostic tool, traditional polysomnography suffers from a key limitation:

Complicated Diagnostic Test to Administer—Because of the large number of sensors and patient connections required for traditional polysomnography, the time and expense associated with setting patients up for the study is high. Sensors must be precisely positioned as improper placement can lead to poor results. Furthermore as patients move throughout the night, sleep techs must continually monitor the signals being received and replace or repair any sensors/wires that become dislodged.

This complexity is a key reason that sleep studies continue to primarily be administered in the sleep lab. The main ramification of this complexity is:

High Cost—Cost of diagnosis by attended overnight polysomnography is estimated to range from $630 to as high as $1000. This cost is partially due to labor and overhead costs associated with the performing the test overnight in a sleep lab. Another significant component of this cost is related to the high cost of capital equipment required for polysomnography. Current full feature polysomnography systems are typically priced around $25,000. Depreciated over the expected life of the equipment, this equates to $53 per test. The electrodes and other disposables currently used account for an additional $58 of cost. The high cost of polysomnography and the fixed capacity of sleep labs retards widespread access to the diagnostic test.

Several competing approaches have been researched for simplified diagnosis of sleep disorders. Systems based simply on overnight oximetry or oximetry in combination with respiration sensors have been developed. These system suffer primarily from their inability to detect whether the patient is asleep. The proposed solution remedies this by performing electrophysiologic measurements required to stage sleep.

Novel measures, such as diagnosis of apnea through heart rate variability analysis or non-invasive monitoring of the peripheral arterial tone have been proposed as well. However, these are not yet widely clinically accepted as diagnostically equivalent to polysomnography. They also suffer from the aforementioned limitation of not being able to detect sleep.

Other approaches to increasing the portability of polysomnographic recording have been proposed, including wireless sensors. These systems address some of the cabling issues but still require the connection of multiple discrete electrodes and are more expensive than the proposed solution due to their wireless transceivers.

Commercially available miniature/portable full polysomnography systems do not integrate the recording electronics onto the sensors at all and thus still utilize multiple individual wires and sensors. Additionally their cost is comparatively high.

The proposed approach will decrease the complexity of the test and thus will lower the cost of evaluating sleep disorders. In addition to enabling screening of sleep disorders outside the sleep lab, the proposed solution will also help sleep labs lower their costs associated with polysomnographic testing. Overall this will result in wider access to such diagnostics testing and improved rate of diagnosis and treatment.

An integrated sensor for making several of the key measurements of polysomnography is the focus of this patent. The development of the sensor will focus on the following aspects to overcome the aforementioned limitations:

Eliminating the wires—The proposed sensor will integrate all amplification and recording electronics onto the sensor substrate. This eliminates the need for wires to be connected between the sensor and the recording equipment. The sensor will store all data on a removable memory cartridge for later review. This also minimizes the need for reattachment of sensors during the study, as there are no wires that may get tangled or dislodge sensors.

Single integrated substrate—Because all sensing elements are integrated on a substrate, application of the sensor is comparatively easier than in traditional polysomnography (where many individual sensors must be placed). Furthermore, sensor placement error is reduced because there is no opportunity to introduce relative positioning errors by placing different sensors incorrectly. Variety in patient size will be handled by the eventual development of several sensors in different sizes

Onboard recording electronics—By positioning the recoding electronics closer to the source of the signal (the sensing element), noise in the recordings is reduced.

The proposed solution thus offers several benefits:

Based on the Principles of Polysomnography

The sensor provides several of the key measurements that are traditionally made in a polysomnography study. This provides a solid basis for the test, as it does not attempt to introduce an alternative means of diagnosis.

Lower Cost

The expected cost of a complete single sensor is less than $25, based on preliminary estimates. This sensor obviates the need for traditional polysomnography equipment ($25,000 capital expense). This lowers the cost per test and removes much of the upfront capital burden on the diagnostic provider. The elimination of the need for any capital equipment greatly enhances the potential of the sensor for eventual use in home apnea screening.

Ease of Use

Because the solution is an integrated self-adhesive sensor with no protruding wires, the ease of use of the system is much simplified. This is expected to enable polysomnography for home apnea screening. Previous systems for home sleep studies have been difficult to use, leading to a higher rate of technical errors and a lack of confidence in the resulting data.

The proposed system will provide a better tool to aid in the diagnosis of sleep apnea. It will help lower costs associated with performing the test, thus promoting greater access to the test and higher rates of diagnosis and treatment of apnea. It will also pave the way for home apnea screening, as it is based on the principles of the current gold standard yet is easy enough and low cost enough to use in the home setting.

### FIGURES

FIG. 1 illustrates one preferred construction for the polysomonographic sensor.

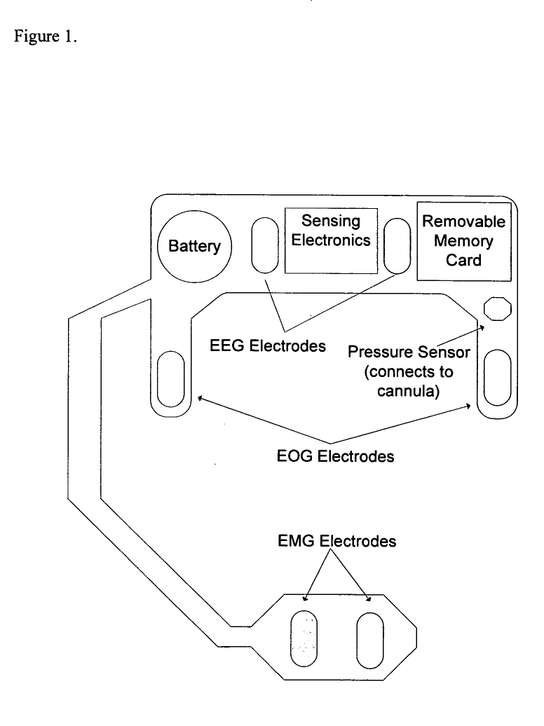

### ALTERATIVE EMBODIMENTS

Holter Monitor

Holter monitoring is a form of ambulatory cardiac monitoring. Patients wear a belt mounted instrument that attaches to several electrodes which are affixed to the patient's torso. The device records electrocardiographic data over an extended (12+ hour) period. The data is later analyzed for irregular heart activity.

Our solution is a Holter sensor which integrates all electrodes and circuitry onto a common flexible substrate. The substrate has an adhesive backing with conductive gel filled wells at the electrode sites. Conductive traces on the substrate interface the electrode sites to the recording and storage electronics. A tiny coin cell battery or thin-film battery is integrated into the device for power. The device is very thin and low-profile and can be comfortably worn under the patient's clothing. No bulky instrument is required, nor are any discrete wires needed. The device contains sufficient onboard memory to record the same data as commercially available instruments. The device may be manufactured such that the control electronics can be easily removed and reused for cost reasons.

Transport/Ambulatory Cardiac Monitoring

Electrocardiography (EKG) is a commonly used diagnostic modality. The traditional way of capturing the EKG is connect several discrete electrodes on a patient's skin to an EKG machine, which is used to sense the electrical signal and display/store/analyze the sensed waveform. In certain instances the wired connection between the electrodes and the machine presents difficulties. Two examples are: 1) Transport monitoring-when a patient is being moved by ambulance, helicopter or stretcher—the wires can interfere with the movement of personnel and become accidentally detached, preventing the acquisition of a valid EKG signal. 2) In the hospital often ambulatory patients must be moved from one place to another (to a testing/imaging lab, for example), but it is desirable to continuously record the EKG. Currently this is difficult as the EKG machine and the electrode leads interfere.

Our solution is an EKG sensor which integrates all electrodes and circuitry onto a common flexible substrate. The substrate has an adhesive backing with conductive gel filled wells at the electrode sites. Conductive traces on the substrate interface the electrode sites to the recording and storage electronics. A tiny coin cell battery or thin-film battery is integrated into the device for power. The device is very thin and low-profile and can be comfortably worn under the patient's clothing. No bulky instrument is required, nor are any discrete wires needed. The device has the ability to wirelessly transmit the sensed EKG signal to a remote monitor in real time. The device can transmit its data to existing EKG machines which are outfitted with a simple adapter to allow them to receive the wireless data. This sensor will provide wireless cardiac monitoring for patients who are being moved. Because the sensor conforms and adheres directly to the patients skin, there are no wires to become dislodged or to interfere with the movement of the patient's caregivers. The device may be manufactured such that the control electronics can be easily removed and reused for cost reasons.

Sleep Sensor

In the diagnosis of sleep disorders, the sleeping state is detected through some combination of EEG, EOG, and EMG. This involves the application of multiple electrodes to a patient's head and face and connection of these electrodes to polysomnographic recording equipment. While this is the gold standard, it can be challenging to maintain proper electrode placement and interconnection while a patient sleeps. In addition it is somewhat uncomfortable for the patient to sleep with multiple wires attached to his face.

Our solution is an integrated sleep sensor which integrates all electrodes and circuitry onto a common flexible substrate. This sensor could contain several EEG channels, 2R/L EOG channels and a sub-mental EMG channel. Alternatively a subset of these channels could be used. The sensor would be totally self contained and would be applied to the patient's face before sleeping. The substrate has an adhesive backing with conductive gel filled wells at the electrode sites. Conductive traces on the substrate interface the electrode sites to the recording and storage electronics. A tiny coin cell battery or thin-film battery is integrated into the device for power. The device is very thin and low-profile and can be comfortably worn while the patient sleeps. No bulky instrument is required, nor are any discrete wires needed. The device has the ability to wirelessly transmit the sensed signals to a remote monitor in real time as well as the ability to store all the recorded data. The device can transmit its data to existing polysomnography machines which are outfitted with a simple adapter to allow them to receive the wireless data. Alternatively the device can transmit its data to a low cost unit which can be interfaced to a personal computer. Because the sensor conforms and adheres directly to the patient's skin, there are no wires to become dislodged or to interfere with the movement of the patient. The device may be manufactured such that the control electronics can be easily removed and reused for cost reasons.

Actigraph/Motion Sensor

An actigraph is a device which records a user's motion, typically by means of an accelerometer. Actigraphs are usually wither wristwatch-like units or are mounted on elastic bands which can be worn on the leg. Actigraphy is commonly used in sleep studies. In these scenarios the actigraph is typically connected, by wire, to a polysomnography machine. Actigraphs may also be used in diet and weight control programs as a means of continuously monitoring the quantity of physical activity that a person engages in.

Our solution is a thin, flexible actigraph which integrates all sensing elements and circuitry onto a common flexible substrate. This sensor could contain one or more accelerometers. The substrate has an adhesive backing which allows the sensor to be easily affixed to the patient's skin. A tiny coin cell battery or thin-film battery is integrated into the device for power. The device is very thin and low-profile and can be comfortably worn while the patient sleep or goes about his/her normal daily activities. No bulky instrument is required, nor are any discrete wires needed. The device has the ability to wirelessly transmit the sensed signals to a remote monitor in real time as well as the ability to store all the recorded data. The device can transmit its data to existing polysomnography machines which are outfitted with a simple adapter to allow them to receive the wireless data. Alternatively the device can transmit its data to a low cost unit which can be interfaced to a personal computer. Because the sensor conforms and adheres directly to the patient's skin, there are no wires to become dislodged or to interfere with the movements of the patient. The device may be manufactured such that the control electronics can be easily removed and reused for cost reasons.

Respiration/Respiratory Effort Sensor by Motion

Monitoring a patient's respiration or respiratory effort is an often performed measurement. One indirect method of doing this is by measuring the excursions of the chest wall. This can be accomplished either through the use of an accelerometer or a strain gauge device. Typical devices that employ this technique would be comprised of a sensor mounted on a band to be worn around the torso. This sensor would then be connected to an external piece of equipment for receiving the sensor output.

Our solution is a thin, flexible device which integrates all sensing elements and circuitry onto a common flexible substrate. This sensor could contain one or more accelerometers or strain gauges. The substrate has an adhesive backing which allows the sensor to be easily affixed to the patient's skin. A tiny coin cell battery or thin-film battery is integrated into the device for power. The device is very thin and low-profile and can be comfortably worn while the patient sleeps or goes about his/her normal daily activities. No bulky instrument is required, nor are any discrete wires needed. The device has the ability to wirelessly transmit the sensed signals to a remote monitor in real time as well as the ability to store all the recorded data. Because the sensor conforms and adheres directly to the patient's skin, there are no wires to become dislodged or to interfere with the movements of the patient. The device may be manufactured such that the control electronics can be easily removed and reused for cost reasons.

Respiration/Respiratory Effort Sensor by Impedance Pneumography (or Inductive Plethysmography)

Monitoring a patient's respiration is an often performed measurement. One method of doing this is through impedance pneumography. This involves placing one or more electrodes over the patient's thorax and monitoring the impedance across the electrodes. As a patient breathes the impedance changes and is detected by recording electronics. Typical devices that employ this technique would be comprised of multiple electrodes placed on the patient which are connected to an external piece of equipment.

Our solution is an impedance pneumography sensor which integrates all electrodes and circuitry onto a common flexible substrate. The substrate has an adhesive backing with conductive gel filled wells at the electrode sites. Conductive traces on the substrate interface the electrode sites to the recording and storage electronics. A tiny coin cell battery or thin-film battery is integrated into the device for power. The device is very thin and low-profile and can be comfortably worn under the patient's clothing. No bulky instrument is required, nor are any discrete wires needed. The device has the ability to wirelessly transmit the sensed signal to a remote monitor in real time. Because the sensor conforms and adheres directly to the patients skin, there are no wires to become dislodged or to interfere with the movement of the patient's caregivers. The device may be manufactured such that the control electronics can be easily removed and reused for cost reasons.

Pulse Oximetry Sensor

Pulse oximetry is a simple non-invasive method of monitoring the percentage of haemoglobin (Hb) which is saturated with oxygen. It involves attaching a sensor to patient's finger, earlobe or forehead. The sensor has a light emitting diode/photodetector pair. Light emitted by the diode is detected by the photodetector after passing through or reflect by the region of interest. The wavelength is chosen such that the amount of received energy can be correlated to the oxygenation level of the blood in the region of interest. Pulse oximetry is widely used in critical care and other patient monitoring settings. The sensor connected to the patient is interface to separate recording instrument which controls the LED and processes the received signals.

Our solution is a pulse oximetry sensor which integrates all electrodes and circuitry onto a common flexible substrate. The substrate has an adhesive backing. A tiny coin cell battery or thin-film battery is integrated into the device for power. The device is very thin and low-profile and can be comfortably worn by the patient. No bulky instrument is required, nor are any discrete wires needed. The device has the ability to wirelessly transmit the sensed signal to a remote monitor in real time. Because the sensor conforms and adheres directly to the patients skin, there are no wires to become dislodged or to interfere with the movement of the patient's caregivers. The device may be manufactured such that the control electronics can be easily removed and reused for cost reasons. We anticipate versions that work on the patient's fingertip, earlobe and forehead.

Bilirubin Sensor

The measurement of bilirubin levels in neonates is a commonly performed diagnostic test to asses jaundice. This is typically done via a blood test. Recently several devices have been introduced which perform this measurement transdermally, by emitting light of a particular frequency into a neonate's forehead and measuring the amount of light that is reflected. While both the direct blood test and the new method are viable, they are not continuous measurements—they provide the bilirubin level at only one point in time. It would be more desirable to provide continuous monitoring of bilirubin so that changes could be detected earlier.

Our solution is a bilirubin sensor which integrates all electrodes and circuitry onto a common flexible substrate. The sensor has an onboard LED and photodetector and measures the bilirubin level through the optical method. The substrate has an adhesive backing and is intended to be attached to a neonate's forehand. A tiny coin cell battery or thin-film battery is integrated into the device for power. The device is very thin and low-profile and can be comfortably worn by the patient. No bulky instrument is required, nor are any discrete wires needed. The device has the ability to wirelessly transmit the sensed signal to a remote monitor in real time. Because the sensor conforms and adheres directly to the patients skin, there are no wires to become dislodged or to interfere with the movement of the patient's caregivers. The device is intended to be worn continuously and provides earlier detection of change sin bilirubin levels. The fact that the device is continuously connected also provides improved detection performance as more averaging and signal processing of the received signal can be performed when compared to the instantaneous optical methods.

The device may be manufactured such that the control electronics can be easily removed and reused for cost reasons. We anticipate versions that work on the patient's fingertip, earlobe and forehead.

Skin Temperature Sensor

Measurement of skin temperature is a commonly performed medical procedure. Skin temperature can be used as a proxy for core body temperature and is important in febrile patients and critical care patients. It has also been proven useful for detecting ovulation in women either for birth control purposes or for pregnancy planning. Many devices are available for measuring body temperature—most are instantaneous in nature and do not provide continuous measurements. In many instances continuous monitoring would provide more useful informational and may lead to more accurate temperature readings.

Our solution is a temperature sensor which integrates all sensing electronics and circuitry onto a common flexible substrate. The substrate has an adhesive backing and includes a thermocouple or other solid-state temperature sensor. A tiny coin cell battery or thin-film battery is integrated into the device for power. The device is very thin and low-profile and can be comfortably worn by the patient. No bulky instrument is required, nor are any discrete wires needed. The device has the ability to wirelessly transmit the sensed temperature to a remote monitor in real time. Because the sensor conforms and adheres directly to the patients skin, there are no wires to become dislodged or to interfere with the movement of the patient's caregivers. The device may be manufactured such that the control electronics can be easily removed and reused for cost reasons. The sensor can be worn continuously.

Nerve Stimulator

The application of electrical impulses through the skin to the nerves and muscles is a common procedure in pain management, rehabilitative medicine and some cosmetic procedures. So called TENS and TEMS units are typically small instrument boxes that connect to one or more patient worn electrodes.

Our solution is a nerve/muscle stimulator which integrates all stimulation electronics and circuitry onto a common flexible substrate. The substrate has an adhesive backing with conductive gel filled wells at the electrode sites. Conductive traces on the substrate interface the electrode sites to the recording and storage electronics. A tiny coin cell battery or thin-film battery is integrated into the device for power. The device is very thin and low-profile and can be comfortably worn under the patient's clothing. No bulky instrument is required, nor are any discrete wires needed. Because the sensor conforms and adheres directly to the patients skin, there are no wires to become dislodged or to interfere with the movement of the patient's caregivers. The device may be manufactured such that the control electronics can be easily removed and reused for cost reasons.

Forehead EEG Sensor for Anesthesia Monitoring

The monitoring of the EEG signal on the forehead is commonly used in depth of anesthesia equipment. One or more patient worn electrodes are connected by wire to an instrument which measures the EEG signal and analyzes it to determine the consciousness of a patient.

Our solution is a n EEG sensor that integrates all detection electronics and circuitry onto a common flexible substrate. The substrate has an adhesive backing with conductive gel filled wells at the electrode sites. Conductive traces on the substrate interface the electrode sites to the recording and storage electronics. A tiny coin cell battery or thin-film battery is integrated into the device for power. The device is very thin and low-profile. The device has the ability to wirelessly transmit the sensed signals to a remote monitor in real time as well as the ability to store all the recorded data. The device can transmit its data to existing patient monitoring machines which are outfitted with a simple adapter to allow them to receive the wireless data. Alternatively the device can transmit its data to a low cost unit which can be interfaced to a personal computer. Because the sensor conforms and adheres directly to the patient's skin, there are no wires to become dislodged or to interfere with the movement of the patient or the patient's caregivers. The device may be manufactured such that the control electronics can be easily removed and reused for cost reasons.

Depth of Anesthesia Monitor

One common way to measure the depth of anesthesia is through stimulating a nerve (median or ulnar) with a specific pattern of stimuli (e.g. “train of four”) and sensing the electrical response to the stimuli. The amplitude of the response can be correlated to the depth of anesthesia. This test would typically be performed manually by using a handheld stimulator and EMG machine and visually observing the response.

Our solution is an anesthesia sensor that integrates all stimulation detection electronics and circuitry onto a common flexible substrate. The substrate has an adhesive backing with conductive gel filled wells at the electrode sites. Conductive traces on the substrate interface the electrode sites to the control, recording and storage electronics. A tiny coin cell battery or thin-film battery is integrated into the device for power. The device is very thin and low-profile. The device has the ability to wirelessly transmit the sensed signals to a remote monitor in real time as well as the ability to store all the recorded data. The device can transmit its data to existing patient monitoring machines which are outfitted with a simple adapter to allow them to receive the wireless data. Alternatively the device can transmit its data to a low cost unit which can be interfaced to a personal computer. Because the sensor conforms and adheres directly to the patient's skin, there are no wires to become dislodged or to interfere with the movement of the patient or the patient's caregivers. The device may be manufactured such that the control electronics can be easily removed and reused for cost reasons.

Heart Rate Monitor

Monitoring of heart rate is one of the most basic medical test that is performed. In addition to its importance in critical care and patient motoring, it is also commonly used by athletes and others involved in fitness programs. The measurement of the hear t rate is usually accomplished through either an EKG or a chest strap with recording electronics. In the former case, multiple electrodes and wires are required. In the latter case the chest strap encircles the torso and may be uncomfortable for athletes actively engage din physical activities. New application of heart rate monitoring are also becoming more common with better understanding of heart rate variability and its implications.

Our solution is a heart rate sensor which integrates all electrodes and circuitry onto a common flexible substrate. The substrate has an adhesive backing with conductive gel filled wells at the electrode sites. Conductive traces on the substrate interface the electrode sites to the recording and storage electronics. A tiny coin cell battery or thin-film battery is integrated into the device for power. The device is very thin and low-profile. The device has the ability to wirelessly transmit the sensed signals to a remote monitor in real time as well as the ability to store all the recorded data. Because the sensor conforms and adheres directly to the patient's skin, there are no wires to become dislodged or to interfere with the movement of the patient or the patient's caregivers. The device is small and far less intrusive than a torso band type device. The device may be manufactured such that the control electronics can be easily removed and reused for cost reasons.

Respiration by direct measurement

Monitoring a patient's respiration is an often performed measurement. The most direct method of doing this is by sensing the expelled breath of the patient. This can be accomplished either through the use of a temperature or pressure sensor that is often interface to the end of an oral/nasal cannula. The sensor is typically interfaced by wire to an external piece of recording equipment.

Our solution is a thin, flexible device which integrates all sensing elements and circuitry onto a common flexible substrate. This sensor could contain one or more temperature or pressure sensors. The device is intended to be interfaced to the output of an oral/nasal cannula. The substrate could have an adhesive backing which allows the sensor to be easily affixed to the patient's skin. A tiny coin cell battery or thin-film battery is integrated into the device for power. The device is very thin and low-profile and can be comfortably worn while the patient sleeps or goes about his/her normal daily activities. No bulky instrument is required, nor are any discrete wires needed. The device has the ability to wirelessly transmit the sensed signals to a remote monitor in real time as well as the ability to store all the recorded data. Because the sensor could conform and adheres directly to the patient's skin, there are no wires to become dislodged or to interfere with the movements of the patient. The device may be manufactured such that the control electronics can be easily removed and reused for cost reasons.

1. A patient-worn, self-contained apparatus for the measurement of physiologic variables comprising
a. a flexible substrate,
b. at least one transducer,
c. a power source, and
d. signal acquisition and processing circuitry.

2. The apparatus of claim 1 wherein said flexible substrate comprises polyimide.

3. The apparatus of claim 1 wherein said flexible substrate comprises polyester.

4. The apparatus of claim 1 wherein at least one of said at least one transducers comprises an electromagnetic transducer.

5. The apparatus of claim 1 wherein at least one of said at least one transducers comprises a hydrogel electrode.

6. The apparatus of claim 1 wherein at least one of said at least one transducers comprises a mechanical transducer.

7. The apparatus of claim 1 wherein at least one of said at least one transducers comprises a strain gauge.

8. The apparatus of claim 1 wherein at least one of said at least one transducers comprises an accelerometer.

9. The apparatus of claim 1 wherein at least one of said at least one transducers comprises an optical transducer.

10. The apparatus of claim 1 wherein at least one of said at least one transducers comprises an electromagnetic energy source and an electromagnetic energy detector.

11. The apparatus of claim 1 wherein at least one of said at least one transducers comprises a chemical transducer.

12. The apparatus of claim 1 wherein said power source comprises a coin cell battery.

13. The apparatus of claim 1 wherein said power source comprises a thin film battery.

14. The apparatus of claim 1 wherein said power source comprises radio frequency receiver circuitry for receiving power from an external radio frequency source.

15. The apparatus of claim 1 wherein said signal acquisition and processing circuitry is configured to acquire and process physiological signals.

16. The apparatus of claim 1 wherein said flexible substrate comprises an adhesive backing for affixing the apparatus to patient tissue.

17. The apparatus of claim 1 further comprising a data storage unit.

18. The apparatus of claim 17 wherein said data storage unit comprises a nonvolatile integrated circuit memory chip.

19. The apparatus of claim 17 wherein said data storage unit comprises a removable memory card.

20. The apparatus of claim 1 further comprising a data transmission unit.

21. The apparatus of claim 20 wherein said data transmission unit comprises a radio frequency transmission circuit.

22. The apparatus of claim 20 wherein said data transmission unit comprises an optical transmission circuit.

23. A physiologic sensing apparatus substantially as disclosed herein.

24. A method for measuring physiologic variables substantially as disclosed herein.

25. A method for measuring physiologic variables, said method comprising:
a. Providing a patient-worn, self-contained apparatus for the measurement of physiologic variables comprising: a flexible substrate, at least one transducer, a power source, and signal acquisition and processing circuitry,
b. Affixing the apparatus to patient tissue, and
c. Acquiring at least one physiologic signal.

26. The method of claim 25 further comprising analysis of said at least one physiologic signal so as to assess physiologic condition.

27. The method of claim 26 wherein said analysis is performed by circuitry onboard said apparatus.

28. The method of claim 26 wherein said analysis is performed by circuitry external to said apparatus.
